(19) United States
(12) Patent Application Publication
(10) Pub. No.: US 2026/0337773 A1
(43) Pub. Date: Oct. 29, 2026

(54) NOVEL CHELATING AND STERICALLY ENCUMBERED LIGANDS AND THEIR CORRESPONDING ORGANOMETALLIC COMPLEXES FOR DEPOSITION OF METAL-CONTAINING FILMS

(71) Applicant: L'Air Liquide, Société Anonyme pour l'Etude et l'Exploitation des Procédés Georges Claude, Paris (FR)

(72) Inventors: Jamie GREER, Cologne (DE); Christian DUSSARRAT, Tokyo (JP)

(21) Appl. No.: 19/112,162

(22) PCT Filed: Sep. 14, 2023
(86) PCT No.: PCT/US2023/032749

§ 371 Date: Mar. 14, 2025

(51) Int. Cl. C23C 16/455; C07C 225/06; C07F 1/02; C23C 16/30; C23C 16/40; H01M 10/42

(52) U.S. Cl. CPC C23C 16/45553; C07C 225/06; C07F 1/02; C23C 16/30; C23C 16/409; C23C 16/45531; H01M 10/4235

(57) ABSTRACT

A ligand L capable of forming a coordinated complex with at least one metal atom, the ligand having a general formula:

N(R₁R₂)—C(R₃R₄)—C(R₅R₆)—N(R₇)—CH₂—C(CR₈R₉R₁₀)═O.

The disclosure describes use of L to form liganded metallic complexes ML with a metal M. The disclosure describes use of ML for vapor deposition processes.

### CROSS REFERENCE TO RELATED APPLICATIONS

This application claims priority U.S. Patent Application No. 63/407,325, filed Sep. 16, 2022, the entire contents of which are incorporated herein by reference.

### TECHNICAL FIELD

Organometallic complexes for deposition of metal-containing films, in particular for use in manufacturing semiconductors and battery electrodes.

### BACKGROUND ART

Lithium-containing thin films which are well-known for their use as surface coating layers of electrode materials in lithium-ion battery applications. Examples of lithium containing thin films include lithium phosphate (LiPO), lithium phosphorus oxynitride (LIPON), lithium borate, lithium borophosphate, lithium fluoride, lithium metal fluoride, lithium metal oxide such as lithium niobate, lithium titanate, lithium zirconium oxides, etc. The absence of silicon is preferred for the formation of some of these materials, especially lithium niobate, lithium titanate, lithium zirconium oxides.

During the first cycles of a lithium-ion battery, the formation of a solid electrolyte interface (SEI) on the anode and/or on the cathode are observed from the decomposition of the electrolyte at the electrolyte/electrode interfaces. A loss of capacity of the lithium-ion battery results from the consumption of lithium. In addition, the SEI layers formed are non-uniform and unstable, cracks and dendrites can appear and lead to thermal runaway. Furthermore the SEI layers also create a barrier potential that makes the intercalation in an electrode more difficult.

The surface coating of electrodes by vapor deposition techniques, such as ALD or CVD, is a first choice method to form an intended solid electrolyte interface thin film, hence avoiding the formation of unstable layers. The decomposition of some components of the liquid electrolyte is thus dramatically reduced, the dissolution of transition elements from the cathode material such as manganese is also avoided. Vapor deposition techniques are suitable for depositing a very thin and conformal film to have the above advantages without the drawback of reduced ionic and electronic conductivities. Lithium-containing thin films are highly promising candidates as protective electrode coatings due to their good conductivity and high electrochemical stability.

Another important application of lithium-containing thin films is in the formation of solid electrolyte materials used in solid-state batteries. Solid-state batteries are solvent-free systems with longer lifetime, faster charger time and higher energy density than conventional lithium-ion batteries. Preventing loss of solid electrolyte elements, especially sulfur, is of crucial importance for their long term performances. Solid electrolyte materials are very air and moisture sensitive, a protective layer that improves their performances and their scalability is needed. They are considered as the next technology step in battery development. In the same logic, solid state microbatteries are being implemented in electronics circuits. Lithium-containing thin-film solid electrolytes such as lithium phosphate, lithium borate and lithium borophosphate are deposited by ALD/CVD techniques. Uniform and conformal lithium-containing thin films can even be obtained on complex architecture like 3D batteries.

Vapor deposition of metal (e.g. Li) containing films/coatings, requires a sufficiently: a) thermally stable and b) volatizable chemical precursor, that is c) capable of depositing the metal via a deposition mechanism. The deposition mechanism can be thermal, chemical, surface catalytic, or other routes. Depositing alloys or combination films often requires two or more chemical precursors that can be used together under compatible conditions. Co-reactants such as an oxidizing agent or reducing agent are often also required or significantly improve the deposition process. Identifying metal containing chemical precursors that meet these and other process/application requirements is an ongoing challenge. Vapor deposition on semiconductors and electrode materials can have substantially different process limitations and criteria, for example temperature and aggregate thermal exposure limitations (“thermal budgets”).

N,N,O-tridentate ligands for metals, such as Lithium, have been characterized in the catalyst field. See, e.g., Lu, Wei-Yi, et al. “Synthesis, characterization, and catalytic activity of lithium complexes bearing NNO-tridentate Schiff base ligands toward ring-opening polymerization of L-lactide.” Polymer 139 (2018): 1-10. These molecules are designed for use in catalyzed reactions and are not suitable for vapor deposition.

US20090136677A1 describes a genus of tridentate beta-ketoiminate molecules suitable for use as vapor deposition precursors.

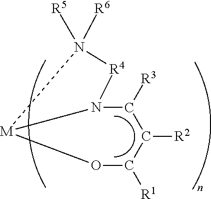

R4 is a C3-10 branched alkylene bridge having at least one chiral carbon atom. This ligand is limited for use with metals having a valence of 2 or more, and thus not for alkali metals such as Na, K or Li.

Several Lithium precursors are known and have been evaluated for their deposition performances. Hämäläinen, Jani, et al. “Lithium phosphate thin films grown by atomic layer deposition.” Journal of The Electrochemical Society 159.3 (2012): A259 https://iopscience.iop.org/article/10.1149/2.052203ies. The two best characterized lithium precursors are lithium tertbutoxide (LiO^{t}Bu) and lithium hexamethyldisilazide [LiHMDS, also known as lithium bis(trimethylsilyl)amide]. LiO^{t}Bu stops forming a quality film at 200 degrees C. and LiHMDS at 250 degrees C.

LiO^{t}Bu has a very low vapor pressure, even at the highest temperatures possible, and the preferred ≥1 torr vapor pressure is not possible. Sønsteby, Henrik H., et al. “tert-butoxides as precursors for atomic layer deposition of alkali metal containing thin films.” Journal of Vacuum Science & Technology A: Vacuum, Surfaces, and Films 38.6 (2020): 060804.

LiHMDS produces vapor deposited materials with both silicon and carbon contaminations well above 1%. Hämäläinen, Jani, et al. “Lithium phosphate thin films grown by atomic layer deposition.” Journal of The Electrochemical Society 159.3 (2012): A259.

There is a particular need for ligands that can form Lithium precursors with better volatility and a vapor deposition temperature below 200 degrees C. Preferably, these Lithium precursors can produce quality vapor deposition films that are conformal with good step coverage, and with low Carbon and Silicon content.

### SUMMARY OF INVENTION

The invention may be understood in relation to the following embodiments presented in the form of numbered sentences:

1. A ligand L capable of forming a coordinated complex with at least one metal atom, the ligand having a general formula:

N(R₁R₂)—C(R₃R₄)—C(R₅R₆)—N(R₇)—CH₂—C(CR₈R₉R₁₀)═O (Formula A)

or

N(R₁R₂)—C(R₃R₄)—C(R₅R₆)—C(R₁₁R₁₂)—N(R₇)—CH₂—C(CR₈R₉R₁₀)═O (Formula B)

and which is structurally represented for Formula A by:

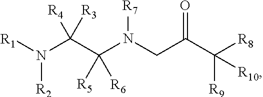

having a SMILES formula:

[R2]N(C[R3])([R4])C([R5])([R6])N(CC(C([R8])([R9])[R10])═O)[R7])[R1],

and which is structurally represented for Formula B by:

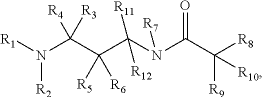

having a SMILES formula:

[R2]N([R1])C([R4])(C([R6])(C([R11])([R12])N([R7])CC(C([R9])([R10])[R8])═O)[R5])[R3]

wherein R₁-R₁₂ are each independently selected from H, a C₁ to C₄ alkyl (linear or branched when C₃ or C₄); or a C₁-C₄ alkyl amino C₁₋₄NR₂ wherein R are each independently selected from H, a C₁ to C₄ alkyl (linear or branched when C₃ or C₄).

2. The ligand L of sentence 1 further comprising a coordinated metal atom M to form a metal-containing chemical ML, structurally represented by:

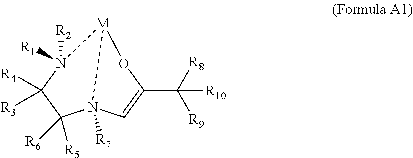

and having a SMILES formula:

[R2][N@@]1([R1])C([R4])(C([R6])([N@]2([R7])C═C(O[M]12)C([R9])([R10])[R8])[R5])[R3],

or structurally represented by:

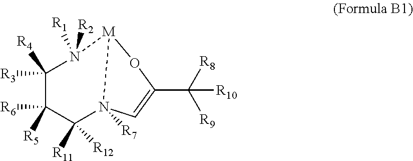

and having a SMILES formula:

[R2][N@]1([R1])[C@]([R4])([C@@]([R6])([C@@]([R11])([R12])[N@]2([R7])CC(C([R9])([R10])[R8])═[O][M]12)[R5])[R3],

wherein M is selected from Li, Na, or K.

3 The metal-containing chemical of sentence 2, wherein M is Li.

4. The ligand of sentence 1 or the metal-containing chemical of sentence 2, wherein R₈, R₉ and R₁₀ are each independently selected from methyl or ethyl.

5. The ligand of sentence 1 or the metal-containing chemical of sentence 2, wherein M is Li and wherein R₈, R₉ and R₁₀ are each independently selected from methyl or ethyl.

6. The metal-containing chemical of sentence 5, wherein R₈, R₉ and R₁₀ are each methyl.

7. The ligand of sentence 1 or the metal-containing chemical of sentence 2, wherein R₁-R₇ are each independently selected from H, methyl, or ethyl.

8 The metal-containing chemical of sentence 7, wherein R₁-R₇ are each independently selected from H or methyl.

9 The metal-containing chemical of sentence 2, structurally represented as:

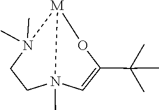

and having a SMILES formula:

CC(C)(C)C1=C[N@@]2(C)CC[N](C)(C)[M]2O1.

10. The metal-containing chemical of sentence 9, wherein M is Li.

11. The metal-containing chemical of sentence 2, wherein M is a polyvalent metal and two or more ligands L are coordinated with M.

12. The metal-containing chemical of sentence 11, wherein M is selected from Calcium, Magnesium, Strontium, Barium.

13. The metal-containing chemical of sentence 2, wherein M is a polyvalent metal and two or more ligands are coordinated with M, wherein M has one or more ligands L and one or more additional different ligands D to form a heteroleptic molecule, M^{x}LyDz, x≥2, y≥1, andz ≥1.

14. The metal-containing chemical of sentence 13, wherein M is selected from Niobium, Tantalum, Vanadium, Zirconium, Hafnium, Titanium, Tungsten, Molybdenum, Chromium, Cobalt, Nickel, Copper, Manganese, Zinc.

15. The metal-containing chemical of sentence 13, wherein M is Niobium.

16. A method of depositing a metal containing film comprising a step of:
a) contacting a substrate with a vapor phase of the metal-containing chemical of any one of sentences 1-15,
b) forming a deposited material on the substrate that comprises the metal from the metal-containing chemical.

17. The method of sentence 16, further comprising a step of exposing the substrate to a gas or vapor phase of one or more additional reactants.

18. The method of sentence 17, wherein M is Li, the deposited material is LiNbOₓ and the one or more additional reactants comprise an Oxygen source reactant and a Niobium source reactant.

19. The method of sentence 17, wherein M is Li, the deposited material is LiPO, and the one or more additional reactants comprise an Oxygen source reactant and a Phosphorus source reactant.

20. The method of sentence 17, wherein M is Li, the deposited material is LiPNO, and the one or more additional reactants comprise an Oxygen source reactant, a Nitrogen source reactant and a Phosphorus source reactant.

21. The method of sentence 18, 19 or 20, wherein the Oxygen source reactant is selected from ozone, hydrogen peroxide, oxygen, water, methanol, ethanol, isopropanol, nitric oxide, nitrous dioxide, nitrous oxide, carbon monoxide, carbon dioxide, and combinations thereof.

22. The method of sentence 18, 19 or 20, wherein the Oxygen source reactant is ozone.

23. The method of sentence 18, wherein the Niobium source reactant is a Group 5 transition metal-containing chemical having one of the following formulae:

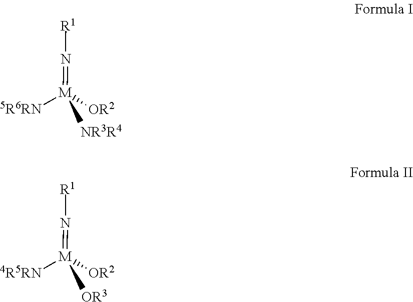

wherein M is Nb and each R¹, R², R³, R⁴, R⁵, and R⁶ is independently selected from H; a C1-C5 linear, branched, or cyclic alkyl group; a C1-C5 linear, branched, or cyclic alkylsilyl group; a C1-C5 linear, branched, or cyclic alkylamino group; or a C1-C5 linear, branched, or cyclic fluoroalkyl group.

24. The method of sentence 18, wherein the Niobium source reactant comprises tert-butylimidobis(diethylamido)mono(tert-butylalkoxo)Niobium(V), tert-butylimidomono(diethylamido)bis(tert-butylalkoxo)Niobium(V), and combinations thereof.

25. The method of sentence 18, wherein the Niobium source reactant is selected from tert-butylimidotris(diethylamido)Niobium(V), tert-butylimidotris(dimethylamido)Niobium(V), tert-butylimidotris(ethylmethylamido)Niobium(V), or a member of the know genus of Nb(RCp)(NR2)₂(═NR) such as tert-butylimidobis(diethylamido)cyclopentadienyl Niobium(V), tert-butylimidobis(dimethylamido)cyclopentadienyl Niobium(V), tert-butylimidobis(dimethylamido) methylcyclopentadienyl Niobium(V), and combinations thereof.

26. The method of sentence 19 or 20, wherein the Phosphorus source reactant is selected from trimethyl phosphate (TMPO), diethyl phosphoramidate (DEPA), triethyl phosphate (TEPO), TMP, and combinations thereof.

27. The method of sentence 19 or 20, wherein the Phosphorus source reactant comprises TMPO.

28. The method of any one of sentences 16-27, wherein step a) and/or step b) is performed at a temperature above a melting point of the metal-containing chemical and at or below 200 degrees C.

29. The method of any one of sentences 16-27, wherein step a) and/or step b) is performed at a temperature above a melting point of the metal-containing chemical and at or below 175 degrees C.

30. The method of any one of sentences 16-27, wherein step a) and/or step b) is performed at a temperature above a melting point of the metal-containing chemical and at or below 150 degrees C.

31. The method of any one of sentences 16-30, wherein a) the substrate has a surface structure with an aspect ratio of 6.25 or less and b) the deposited material on the substrate that comprises the metal from the metal-containing chemical has a step coverage for the surface structure of 50% or more, such as 55%, 60%, 65%, 70%, 75%, 80%, 85%, 90%, 95% or >99%.

32. The method of any one of sentences 16-31, wherein the method comprises an atomic layer deposition (ALD) in which steps a) and b) are repeated in an ALD cycle.

33. The method of claim 32 wherein a growth rate of the deposited material per ALD cycle is 0.2 angstroms of greater, such as 0.3 or greater, 0.4 or greater, 0.5 or greater, 0.6 or greater, 0.7 or greater, 0.8 or greater, 0.9 or greater, 1.0 or greater, 1.1 or greater, or 1.2 or greater.

### BRIEF DESCRIPTION OF DRAWINGS

For a further understanding of the nature and objects for the present invention, reference should be made to the following detailed description, taken in conjunction with the accompanying drawings, in which like elements are given the same or analogous reference numbers and wherein:

FIG. 1 shows TGA results for Example 1;

FIG. 2 shows TGA results for Example 6;

FIG. 3 shows the effect of temperature on deposition rates for Example 7;

FIG. 4 shows the effect of temperature on refractive index of deposited materials for Example 7; and

FIG. 5 shows the effect of temperature on atomic composition of deposited materials for Example 7.

### DESCRIPTION OF PREFERRED EMBODIMENTS

The inventors have synthesized and tested a new tridentate N,N,O ligand for metals that forms a low melting point solid, with good vaporization due to thermal stability, and high volatility; making these liganded metals suitable for vapor deposition. The ligand structure in one genus embodiment is represented by the following formula and structural representations:

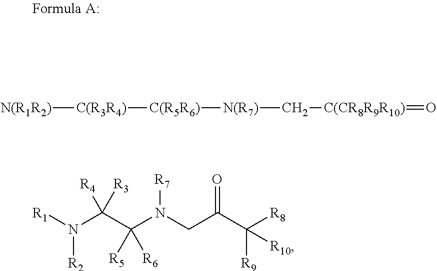

SMILES formula:

[R2]N(C([R3])([R4])C([R5])([R6])N(CC(C([R8])([R9])[R10])═O)[R7])[R1]

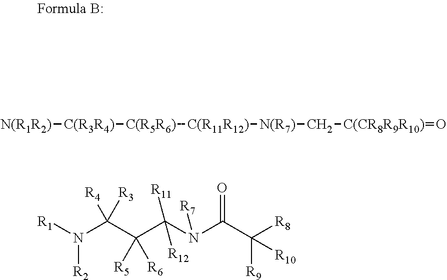

SMILES formula:

[R2]N([R1])C([R4])(C([R6])(C([R11])([R12])N([R7])CC(C([R9])([R10])[R8])═O)[R5])[R3]

R₁-R₁₂ are each independently selected from H, a C₁ to C₄ alkyl (linear or branched when C₃ or C₄); or a C₁-C₄ alkyl amino C₁₋₄NR₂ wherein R are each independently selected from H, a C1 to C4 alkyl (linear or branched when C₃ or C₄).

This new ligand genus is capable of forming a tridentate chelating ligand (“L”) with metal ions. In one preferred embodiment, the metal (“M”) is a monovalent metal ion, such as an alkali metal (e.g. Na⁺ and Li⁺), which forms a chelation chemical ML.

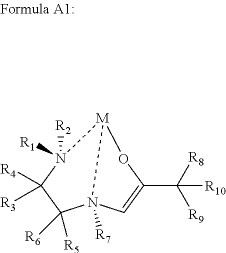

SMILES formula:

[R2][N@@]1([R1])C([R4])(C([R6])([N@]2([R7])C═C(O[M]12)C([R9])([R10])[R8])[R5])[R3]

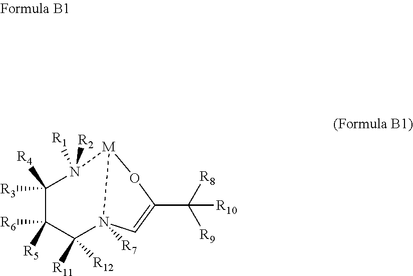

SMILES formula:

[R2][N@]1([R1])[C@]([R4])([C@@]([R6])([C@@]([R11])([R12])[N@]2([R7])CC(C([R9])([R10])[R 8])═[O][M]12)[R5])[R3],

The tridentate ligand L is however capable of chelating higher valency metals alone (M^{+x}Lₓ) or in combinations with other metal ligands (D) to create heteroleptic liganded metals (M^{+x}L_{y}D_{z}). For example, M may be Ca⁺² chelated by two ligands L to form Ca⁺²L₂ (see Example X below) or with a different ligand to form D-Ca⁺²-L.

Chemicals formed with a monovalent metal and the ligand L (ML), generally have a melting point below 150 degrees C, and are thus low melting point solids. Preferred species have a melting point below 70 degrees C. Liquid chemicals, or low melting solids that form a liquid upon heating, are preferred as chemical precursors for vapor deposition processes. Solids that sublimate are also used, but the handling and management of sublimation precursors is more complicated and often associated with higher losses of the chemical due to thermal degradation.

Also highly relevant to use in vapor deposition processes is the vapor pressure achievable with a chemical precursor. This parameter correlates with the speed and quality of the deposited M containing material. Vapor pressure is generally counter trending versus thermal stability. It is important to avoid using chemical precursors that cannot achieve an adequate vapor pressure without using a temperature that also causes substantial thermal degradation of the chemical precursor. These countervailing parameters are generally assessed using thermogravimetric analysis (“TGA”). See, e.g., ASTM E2008-17 (2021), Standard Test Methods for Volatility Rate by Thermogravimetry. A TGA evaluation for vapor deposition precursors assesses the temperature profile of the volatilization curve to define a 50% point at which half the chemical has evaporated (“T₅₀”) and a full vaporization temperature (“T_{full}”) at which further temperature increases do not reduce the weight of residue material. Often this is done at both atmospheric pressure and under a vacuum (e.g. 15 torr), to represent the two common vapor deposition process conditions. TGA also enables a simple evaluation of thermal stability by weight of residual material that does not further vaporize with increased temperature. Finally, vapor pressure produced may be plotted against the temperature/weight curve to have a working evaluation of the three parameters, volatility, T₅₀ and thermal stability.

For vapor deposition processes, it is preferred to have a T₅₀ as low as possible, and as low as possible of a temperature at which the chemical precursor vapor pressure achieves 1 torr (“T₁ₜₒᵣᵣ”). In particular the 1 torr vapor pressure should be at a temperature at which there is as little thermal degradation as possible. (It is this balancing of parameters that is more challenging for sublimated solids.)

Chemicals formed with a monovalent metal and the ligand L (ML), generally have a T₁ₜₒᵣᵣ at or below 150 degrees C. and above the melting point temperature. These same chemicals generally exhibit good vaporization with a) a T₅₀ at or below 250 and above the melting point temperature; and b) a TGA residue of less than 1% by weight.

Due to the above properties, metals M complexed with the ligand L are especially well suited for use in coating cathode electrode materials that include catalyst carbon support structures, such as a single wall fullerene (Ceo and C72), multiwall fullerenes, single wall or multiwall nanotubes, nanohorns, and/or carbon support structures having a density of about 0.2 g/cm3 to about 1.9 g/cm3 such as specialty carbons like VULCAN or Imerys' SUPER C65.

Because of these foregoing properties, chemicals formed with a monovalent metal and the ligand L (ML) are well suited for use in vapor depositions processes that require low temperatures due to substrate limitations on temperature and/or coreactant exposure. LiTHD and LiO^{t}Bu have 1 Torr pressure in excess of 150 degrees C. making for longer pulses and/or higher dep T which are detrimental to many electrode materials in terms of the absolute temperature exposure and also the thermal budget for these materials. Cathode materials for example can undergo lattice changes, oxygen loss, composition change, etc. at higher temperatures of 200 degrees or more. For deposited materials that are negatively affected by Silicon contamination, the ligands L provide a Silicon free chemical precursor capable of depositing M containing films without Si contamination. This is an advantage over LiTMSO and LiHMDS, both of which yield Silicon contaminated deposits, which is particularly a problem at the lowest ALD temperatures available for these chemicals.

Metals M chelated or otherwise coordinated with the ligands L described herein, may be used as M source vapor phase precursors for vapor phase depositions of M containing materials on a substrate. Vapor phase depositions such as chemical vapor deposition and atomic layer deposition are well known in the art. The deposited materials can also contain other atoms from other co-reactants or vapor phase precursors. Common co-reactants are Oxygen source reactants and Phosphorus source reactants and Nitrogen source reactants that, depending on the vapor phase deposition process, can either provide O, N, or P as a dopant, or react with the ML vapor phase precursor to form materials such as oxides or nitides of M, e.g., MOx, MNX, MONx, MPO, MPON, etc. Particular examples herein are LiNbOx and LiPOx, but many other materials are possible with different combinations of precursors and co-reactants. One of skill in the art can select from a variety of known Oxygen source reactants, Phosphorus source reactants and Nitrogen source reactants to design a vapor phase deposition process. Oxygen source reactants include O₂, O₃, H₂O, H₂O₂, NO, NO₂, a carboxylic acid, an alcohol, a diol, radicals thereof, and combinations thereof. Nitrogen source reactants include N₂, H₂, NH₃, hydrazines (such as N₂H₄, MeHNNH₂, MeHNNHMe), organic amines (such as NMeH₂, NEtH₂, NMe₂H, NEt₂H, NMe₃, NEt₃, (SiMe₃)₂NH), pyrazoline, pyridine, diamines (such as ethylene diamine), radical species thereof, and mixtures thereof. Phosphorus source reactants include trimethyl phosphate (TMPO), diethyl phosphoramidate (DEPA), triethyl phosphate (TEPO), TMP, and combinations thereof.

### EXAMPLES

### Example 1—Synthesis

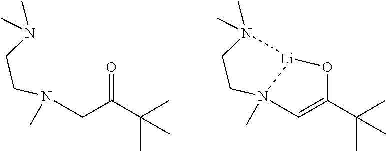

O═C(tBu)CH2NMe(CH2)2NMe2 Li—O—C(tBu)=CHNMe(CH2)2NMe2

O═C(tBu)CH2NMe(CH2)2NMe2: ligand synthesized by 1:1 reaction of O═C(tBu)CH2Cl and HNMe[(CH2)2NMe2 at 50-60 °C in THF/ACN with TEA, K2CO3, or NaHCO3. 1H NMR (CDCl3, 400 MHz): 3.412 (2H, br s), 2.488 (2H, t, 3J=7.7 Hz), 2.327 (2H, t, 3J=7.7 Hz), 2.247 (3H, br s), 2.147 (6H, br s), 1.060 (9H, br s)

The complex was prepared by reaction of O═C (tBu)CH2NMe[(CH2)2NMe2 with BuLi, LiNH2, or LiH, in either hexane, pentane, or MTBE. The crude material was purified by distillation to produce a white solid with a yield of 85%. Melting Point=68 degrees C.

1H NMR (C6D6, 400 MHz): 2.110 (6H, br s), 1.871 (2H, q, 3J=10.2 Hz), 1.625 (2H, q, 3J=10.2 Hz), 2.811 (1H, t, 3J=11.9 Hz), 2.460 (1H, t, 3J=11.9 Hz), 2.345 (3H, s), 4.126 (1H, s), 1.369 (9H, s) 13C NMR (C6D6, 101 MHz): 57.1, 46.9, 36.2, 57.5, 102.6, 171.3, 30.3, 28.9.

TGA was carried out under the following measurement conditions: sample weight of 11.4 mg, closed cup under N2 at 1 atm (full evaporation temperature at 305 degrees C); sample weight of 10.4 mg, open cup under N2 at 1 atm (full evaporation temperature T_{full}=254 degrees C); sample weight of 30.1 mg, open cup under N2 at 15 torr (full evaporation temperature T_{full}=178 degrees C). Rate of temperature increase set to 10.0 °C/min. 50% evaporation T₅₀ using open cup TGA method under N2 at 1 atm=225 degrees C.

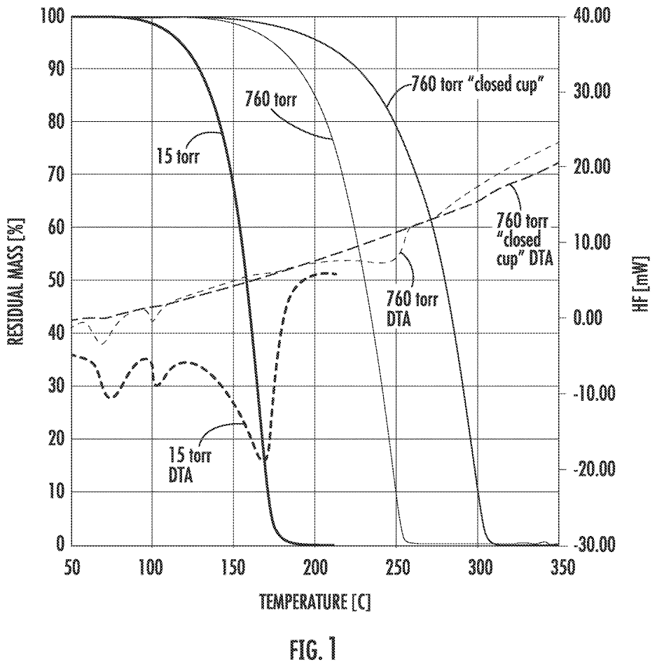

These results are graphically presented in FIG. 1. As shown, at T_{full} the residue is negligible (less than 1% by weight).

### Examples 2-5—Additional Syntheses

The same synthesis was performed with the following variations of Example 1:
Example 2: Positions R1, R2 and R7 were changed to ethyl
Example 3: Position R7 was changed to n-butyl
Example 3: Position R7 was changed to ethyl
Example 5: M was Na

Synthesis of ML was performed in the same manner as Example 1, with L having the different alkyl groups noted above. For M=Na, the Na was provided as NaNH2 for formation of ML. Similar results were obtained in terms of yield and purity.

### Example 6—Synthesis of Exemplary M⁺²L₂, M=Ca

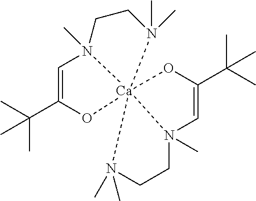

Synthesis: Na{O—C(tBu)═CHN(Me)(CH₂CH₂NMe₂)} added to 0.5 equivalents Cal₂ in THF and stirred for 16 hrs. Filtered and sublimed (120 degrees C/0.1 Torr) 1H NMR (C6D6, 400 MHz): 3.812 (2H, br s), 2.488 (4H, br dt), 2.327 (4H, br dt), 2.147 (6H, br s), 2.032 (12H, br s), 1.000 (19H, br s).

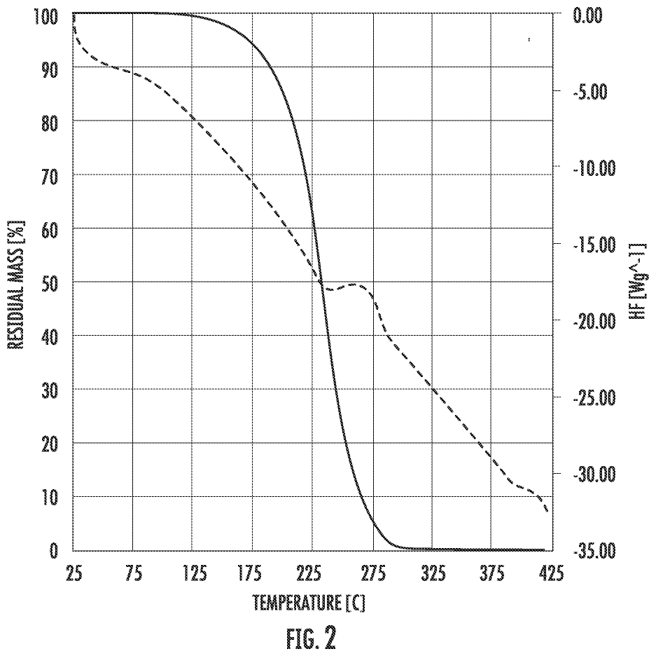

TGA measurement was carried out under the following measurement conditions: sample weight of 6.2 mg, open cup under N2 at 1 atm (full evaporation temperature at 280 degrees C) Rate of temperature increase set to 10.0 °C/min. 50% evaporation using open cup TGA method under N2 at 1 atm; T₅₀=230 degrees C, as shown in FIG. 2.

### Example 7—LiPOx Deposition on a Substrate

The Li—O—C(tBu)═CHNMe(CH2)2NMe2 from Example 1 was used as the Li source for an atomic layer deposition (ALD) of a LiPOx film on a blank silicon wafer as the test substrate. Trimethyl phosphate (TMPO) is an art standard Phosphate source used with prior art Li precursors. We therefore selected TMPO as the co-reactant.

### Preliminary Characterizations:

Li—O—C(tBu)═CHNMe(CH2)2NMe2 thermally decomposes at 250 degrees C. The baseline design of experiment parameter evaluations were therefore done at 200 degrees C to avoid parasitic CVD.
Pulse time dosage escalation of both precursors demonstrated self-limited growth at 0.9 angstroms/cycle under the conditions tested (ozone as a co-reactant). This confirmed the 200 degree deposition was an ALD process.
An Oxygen source was required to form the LiPOx. No ALD occurred in the absence of Ozone.

The design of experiment parameters from the 200 degree c preliminary tests were then used to evaluate the effect of temperature on ALD of LiPOx.

### Experimental Conditions:

| Reactor temperature: | X | °C |
|---|---|---|
| Reactor Pressure: | 1 | torr |
| Carrier N₂: | 80 | sccm |
| canister T: | 110 | °C |
| canister P: | 30 | torr |
| N₂ in Li bubbling FR: | 30 | sccm |
| TMPO canister T: | 80 | °C |
| TMPO canister P: | 15 | torr |
| N₂ in P bubbling FR: | 30 | sccm |

| | |
|---|---|
| Substrate: | Si (1% HF) |
| Number of cycles: | 150 |

### Pulse Conditions:

| | | |
|---|---|---|
| Li—O—C(tBu)═CHNMe(CH2)2NMe2 (0.64 sccm): | 30 | s |
| Purge: | 120 | s |
| TMPO (30 sccm): | 15 | s |
| Purge: | 30 | s |
| O₃: | 5 | s |
| Purge: | 30 | s |

As shown in FIGS. 3-5, the temperatures tested were 125, 150, 175 and 200 degrees C. LiPOx deposited across this temperature range as a uniform continuous film.

Per FIG. 3, the main difference (as one would expect) was rate of deposition, with maximum deposition rates at 200 degrees C. However, even at 125 degrees C, each cycle of ALD formed a 0.36 angstrom thick deposit, which is a commercially viable deposition rate. This is in contrast to the prior art molecules discussed above for which deposition ceases at or above 200 degrees C.

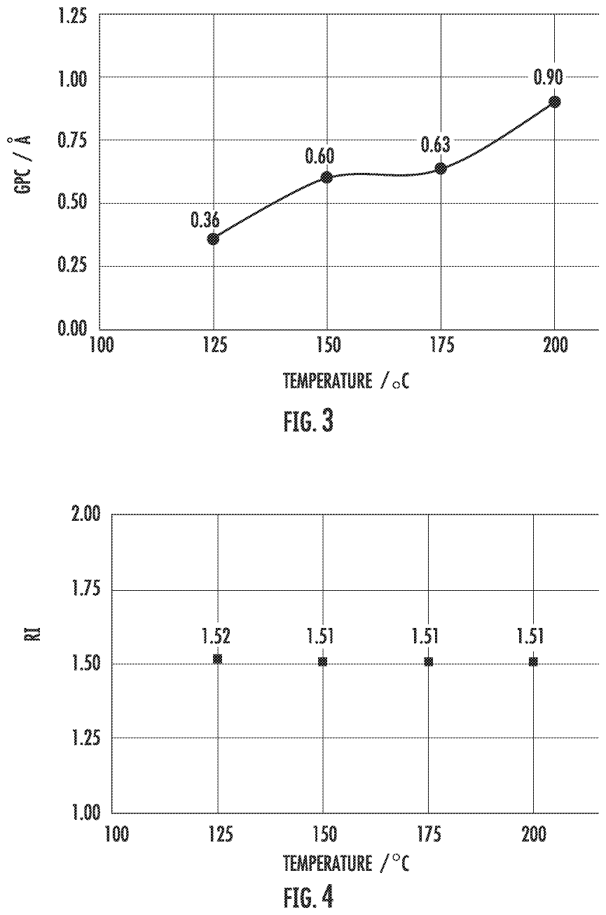

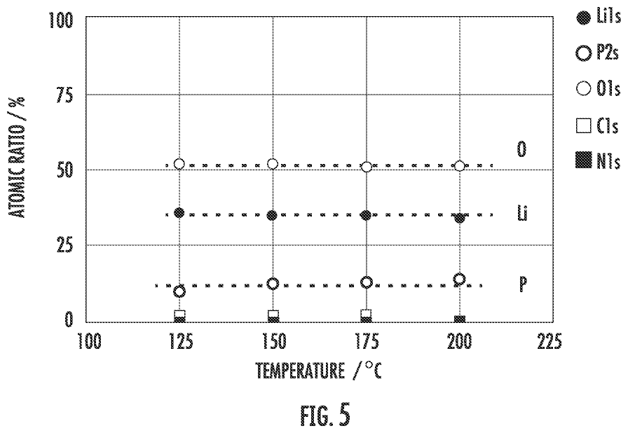

FIG. 4 shows that the refractive index of the deposited material remains stable across the temperature range and is close in value to Li₃PO₄ (RI 1.59).

FIG. 5 shows the atomic percentage, with Silicon, N and C both below the limit of detection (which is lower than 1%). Consistent with the RI measurements, the deposited material has a composition of Li_{2.8}PO_{3.8}, very close to Li₃PO₄. This combination of low temperature ALD, GPC rate and composition is an important advance that will enable LiPOx depositions on a variety of temperature/thermal budget restricted substrates, especially materials used for Li ion battery electrodes.

Good step coverage of non-uniform substrate surfaces is important to good electrode/cathode performance because 1) thicker points will inhibit Li ion transport 2) thinner points will result in TM loss (e.g. Mn migration out of cathode) and/or dendrite formation. To evaluate the applicability of this ALD process to nonuniform surfaces, a test Si wafer with trenches was used as a substrate. At an aspect ratio of 6.25, the step coverage was 87%. The maximum aspect ratio tested was 18. Even at this severe aspect ratio, the ALD layer was continuous. The step coverage at this dimension was 65%, indicating that the ALD deposition process is attenuated on extremely remote surfaces. Nonetheless, the minimum thickness achieved in the trench bottom was 14.9 nm, which is adequate for many applications. In many applications, such as battery electrode materials, step coverage less than 100% is acceptable. We expect with further parameter optimization, these results will be improved compared to these preliminary experiments.

### Example 8—LiNbOx Deposition on a Substrate

As a diversified material deposition example, we selected a binary deposition for LiNbO₃. The Li—O—C(tBu)═CHNMe(CH2)2NMe2 of Example 1 was used as a Li source precursor. For Niobium, we selected the source precursor tert-butylimidobis(diethylamido)mono(tert-butylalkoxo)Niobium(V) described in U.S. Pat. No. 10,106,88782. One reason to select tert-butylimidobis(diethylamido)mono(tert-butylalkoxo)Niobium(V) was that it has an ALD temperature window for NbOx deposition that extends at least to as low as 150 degrees C. A temperature of 175 degrees C was selected to balance the Li and Nb source precursors' ALD windows and GPC. Again a blank silicon wafer was used as the substrate for preliminary evaluations. The process conditions tested were:

| | | |
|---|---|---|
| T [°C] (set, actual) | 175 | |
| P_{reactor} [Torr] | 1 | |
| T_{can}[°C], VP_{prec}. [Torr] | 115 | 1 |
| P_{can} [Torr] | 30 | |
| Carrier [sccm] | 15 | N₂ |
| FR [sccm], PP [Torr] | 0.64 | |
| Coreactant | O₃ | |
| FR [sccm], PP [Torr] | 50 | |
| Dil FR [sccm] | 200 | N₂ |

The ALD cycle parameters were:

| Pulses [s] | |
|---|---|
| # Cycles | 300 |
| Li precursor | 40 |
| Purge | 90 |
| O₃ pulse | 10 |
| Purge | 15 |
| Nb precursor | 30 |
| Purge | 60 |
| O₃ pulse | 10 |
| Purge | 15 |

The Nb-ozone subcycles form layers of NbOx. The Lithium precursor reacts with this NbOx to form a LiNbOx material. The aggregate deposition growth rate for LiNbOx is 0.68 angstroms. Pulse dose experiments confirmed that the LiNbOx formation is a self-limiting ALD reaction. The stoichiometry of the deposited material was approximately LiNbO₂. This is consistent with the RI of 1.9 for the deposited material compared to 2.2 for LiNbO₃. The deposition was further tested on a silicon wafer with trenches. Step Coverage was >99% even at an aspect ratio of 15. The deposited layer was continuous and free of gaps or visible defects in slices scanned by SEM. It is expected that, with further parameter optimizations (such as ozone dosing), the deposited material will be very close in atomic composition to LiNbO₃, similar to the results ultimately obtained for LiPOx.

### Example 9—Synthesis on Exemplary M^{x}LyDz

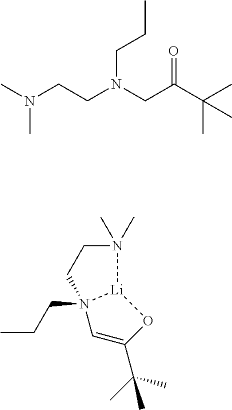

^{t}BuN=Nb(OEt)₂{O—C(^{t}Bu)=CHN(Me)(CH₂CH₂NMe₂)}

Synthesis: ^{t}BuN=NbCl₃ added to 2 equiv. NaOEt and 1 equiv. of Li{O—C(^{t}Bu)═CHN(Me)(CH₂CH₂NMe₂)} in THF and stirred over 16 hrs. Filtered and purified via distillation (110 degrees C/0.1 torr) ¹H NMR (C₆D₆, 400 MHz): 4.561 ppm (4H, tm), 4.002 ppm (1H, s), 2.610 ppm (1H, t, 3J=11.6 Hz), 2.334 ppm (1H, d, 2J=14.7 Hz), 2.382 ppm (3H, s), 2.108 ppm (3H, s), 1.975 ppm (3H, s), 1.323 ppm (1H, d, 3J=14.7 Hz), 1.239 ppm (1H, t, 3J=11.6 Hz), 1.163 ppm (6H, m), 1.108 ppm (9H, s), 1.006 ppm (9H, s).

### Example 9: Synthesis of Li—OC(^{t}Bu)═CHN[(CH₂)₂OMe]₂

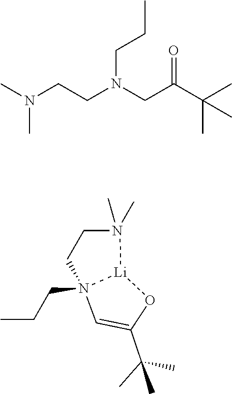

O═C(tBu)CH2N[(CH2)3NMe2]2 (left); Li—OC(tBu)=CHN[(CH2)3NMe2]2 (right)

O═C(tBu)CH2N[(CH2)3NMe2]2: ligand synthesized by 1:1 reaction of O═C(tBu)CH2Cl and HN[(CH2)3NMe2]2 at 50-60 degrees C in THF/ACN with TEA, K2CO3, or NaHCO3.

¹H NMR (CDCl3, 400 MHz): 2.144 ppm (12H, s), 2.193 ppm (4H, t, 3J=7.2 Hz), 1.541 ppm (1H, p, 3J=7.2 Hz), 2.475 ppm (4H, t, 3J=7.2 Hz), 3.465 ppm (2H, s), 1.075 ppm (9H, s) The complex was prepared by reaction of O═C(tBu)CH2N[(CH2)3NMe2]2 with BuLi, LiNH2, or LiH, in either hexane, pentane, or MTBE. The crude material was purified by sublimation to produce a yellow solid Yield (45%). ¹H NMR (C6D6, 400 MHz): 4.155 ppm (1H, br s), 2.495 ppm (4H, br s), 2.188 ppm (12H, br s), 2.1-2.3 ppm (4H, br m), 2.320 ppm (2H, br d, 3J=11.1 Hz), 1.400 ppm (9H, br s)

¹³C NMR (C6D6, 101 MHz): 173.6, 98.8, 70.6, 58.4, 57.9, 36.4, 29.3.

### Example 10—Synthesis of Li—OC (^{t}Bu)═CHN[(CH₂)₂OMe]₂

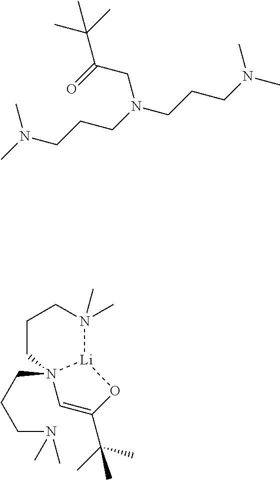

O═C(^{t}Bu)CH₂N[(CH₂)₃NMe₂]₂ (left); Li—OC(^{t}Bu)═CHN[(CH2)₃NMe₂]₂ (right)

O═C(^{t}Bu)CH₂N[(CH₂)₃NMe₂]₂: ligand synthesized by 1:1 reaction of O═C(^{t}Bu)CH₂Cl and HN[(CH₂)₃NMe₂]₂ at 50-60 degrees C in THF/ACN with TEA, K₂CO₃, or NaHCO₃.

¹H NMR (CDCl3, 400 MHz): 2.144 ppm (12H, s), 2.193 ppm (4H, t, 3J=7.2 Hz), 1.541 ppm (1H, p, 3J=7.2 Hz), 2.475 ppm (4H, t, 3J=7.2 Hz), 3.465 ppm (2H, s), 1.075 ppm (9H, s) The complex was prepared by reaction of O═C(ₜBu)CH₂N[(CH₂)₃NMe₂]₂ with BuLi, LiNH₂, or LiH, in either hexane, pentane, or MTBE. The crude material was purified by sublimation to produce a yellow solid Yield (45%).

¹H NMR (C6D6, 400 MHz): 4.155 ppm (1H, br s), 2.495 ppm (4H, br s), 2.188 ppm (12H, br s), 2.1-2.3 ppm (4H, br m), 2.320 ppm (2H, br d, 3J=11.1 Hz), 1.400 ppm (9H, br s).

¹³C NMR (C6D6, 101 MHz): 173.6, 98.8, 70.6, 58.4, 57.9, 36.4, 29.3.

### INDUSTRIAL APPLICABILITY

The present invention is at least industrially applicable to chemical precursors suitable for use in deposition of material for semiconductor manufacture or battery electrodes.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

All references identified herein are each hereby incorporated by reference into this application in their entireties, as well as for the specific information for which each is cited.

### LEGAL DEFINITIONS AND INTERPRETATIONAL PRINCIPLES

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

Reference herein to “one embodiment” or “an embodiment” means that a particular feature, structure, or characteristic described in connection with the embodiment may be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used herein, “about” or “around” or “approximately” in the text or in a claim means±10% of the value stated.

### TECHNICAL DEFINITIONS

As used herein, “room temperature” in the text or in a claim means from approximately 20 °C to approximately 25 °C.

The term “ambient conditions” refers to an environment temperature (i.e., ambient temperature) approximately 20 °C to approximately 25 °C and an environment pressure (ambient temperature) approximately 1 atm or 1 bar.

The term “substrate” refers to a material or materials on which a process is conducted. The substrate may also have one or more layers of differing materials already deposited upon it from a previous manufacturing step.

One of ordinary skill in the art will recognize that the terms “film” or “layer” used herein refer to a thickness of some material laid on or spread over a surface and that the surface may be a trench or a line.

The standard abbreviations of the elements from the periodic table of elements are used herein. It should be understood that elements may be referred to by these abbreviations (e.g., Si refers to silicon, N refers to nitrogen, O refers to oxygen, C refers to carbon, H refers to hydrogen, F refers to fluorine, etc.).

The unique CAS registry numbers (i.e., “CAS”) assigned by the Chemical Abstract Service are provided to help better identify the molecules disclosed.

The Aspect Ratio of a geometric shape is the ratio of its sizes in different dimensions. The aspect ratio is most often expressed as two integer numbers separated by a colon (x:y). The values x and y do not represent actual widths and heights but, rather, the proportion between width and height. As an example, 8:5, 16:10, 1.6:1 are all ways of representing the same aspect ratio. In objects of more than two dimensions, such as hyperrectangles, the aspect ratio can still be defined as the ratio of the longest side to the shortest side.

Conformality and Step Coverage both refer to the degree of variability in the thickness of a film on a surface, especially topologically different areas of a surface. This is especially relevant to surfaces with microstructures having various aspect ratios. Complete (100%) conformality for the above example means there is zero cusping and the top surface, the trench sidewall and when applicable the trench bottom, have all identical thicknesses. If a single conformality percentage is given, it is the least conformal measurement corresponding to the greatest deviation in relative thickness of the overall film at two selected points on the surface. The two points may correspond to the highest Aspect Ratio points or, for example, points having a specific Aspect Ratio such as 6:1 or less. Thicknesses of films are assessed by a number of methods, for example scanning electron microscopy of sectioned substrates. A film is generally “conformal” if the film is at least 20% conformality, preferably at least 50%.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1.-20. (canceled)

21. A ligand L capable of forming a coordinated complex with at least one metal atom, the ligand having a general formulae:
N(R₁R₂)—C(R₃R₄)—C(R₅R₆)—N(R₇)—CH₂—C(CR₈R₉R₁₀)═O (Formula A),
which is structurally represented by: 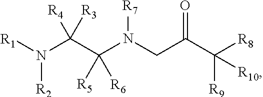
or N(R₁R₂)—C(R₃R₄)—C(R₅R₆)—C(R₁₁R₁₂)—N(R₇)—CH₂—C(CR₈R₉R₁₀)═O (Formula B), which is structurally represented by: 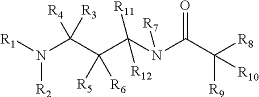
wherein R₁-R₁₂ are each independently selected from H, a C₁ to C₄ alkyl (linear or branched when C₃ or C₄); or a C₁-C₄ alkyl amino C₁₋₄NR₂ wherein R are each independently selected from H, a C1 to C4 alkyl (linear or branched when C₃ or C₄).

22. The ligand of claim 1, further comprising a coordinated metal atom M to form a metal-containing chemical, structurally represented by: 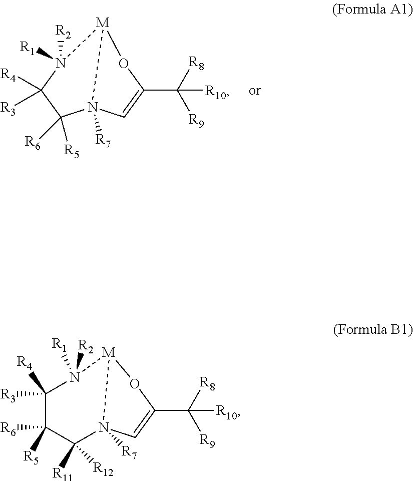
wherein M is selected from Li, Na, or K.

23. The metal-containing chemical of claim 2, wherein M is Li.

24. The ligand of claim 1, wherein R₈, R₉ and R₁₀ are each independently selected from methyl or ethyl.

25. The metal-containing chemical of claim 2, wherein R₈, R₉ and R₁₀ are each independently selected from methyl or ethyl.

26. The metal-containing chemical of claim 2, wherein M is Li and wherein R₈, R₉ and R₁₀ are each independently selected from methyl or ethyl.

27. The metal-containing chemical of claim 2, wherein M is Li and wherein R₈, R₉ and R₁₀ are each independently selected from methyl or ethyl.

28. The metal-containing chemical of claim 5, wherein R₈, R₉ and R₁₀ are each methyl.

29. The ligand of claim 1, wherein R₁-R₇ are each independently selected from H, methyl, or ethyl.

30. The metal-containing chemical of claim 2, wherein R₁-R₇ are each independently selected from H, methyl, or ethyl.

31. The metal-containing chemical of claim 10, wherein R₁-R₇ are each independently selected from H or methyl.

32. The metal-containing chemical of claim 2, structurally represented as:

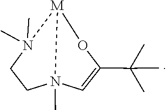

33. The metal-containing chemical of claim 12, wherein M is Li.

34. The metal-containing chemical of claim 2, wherein M is a polyvalent metal and two or more ligands L are coordinated with M.

35. The metal-containing chemical of claim 2, wherein M is a polyvalent metal and two or more ligands with M, wherein M has one or more ligands L and one or more additional different ligands D to form a heteroleptic molecule, M^{x}LyDz, x≥2, y≥1, and z≥1.

36. A method of depositing a metal containing film comprising a step of:
a) contacting a substrate with a vapor phase of the metal-containing chemical of claim 2,
b) forming a deposited material on the substrate that comprises the metal from the metal-containing chemical.

37. The method of claim 16, further comprising a step of exposing the substrate to a gas or vapor phase of one or more additional reactants.

38. The method of claim 17, wherein M is Li, the deposited material is LiNbOx and the additional reactants comprise an Oxygen source reactant and a Niobium source reactant.

39. The method of claim 17, wherein M is Li, the deposited material is LiPO or LiPON, and the additional reactants comprise an Oxygen source reactant and a Phosphorus source reactant, and for LiPON the additional reactants further comprise a Nitrogen source reactant.

40. The method of claim 18, wherein the oxygen source reactant is ozone.

41. The method of claim 19, wherein the oxygen source reactant is ozone.

42. The method of claim 18, wherein the Niobium source reactant comprises a Group 5 transition metal-containing chemical having one of the following formulae: 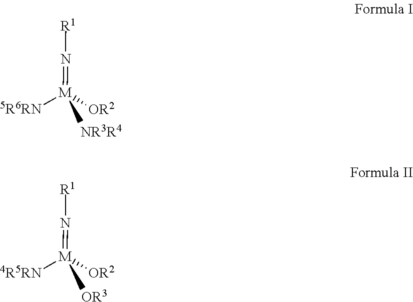
wherein M is Nb and each R¹, R², R³, R⁴, R⁵, and R⁶ is independently selected from H;
a C1-C5 linear, branched, or cyclic alkyl group; a C1-C5 linear, branched, or cyclic alkylsilyl group; a C1-C5 linear, branched, or cyclic alkylamino group; or a C1-C5 linear, branched, or cyclic fluoroalkyl group; or Nb(RCp)(NR2)2(═NR) (Formula III); or selected from tert-butylimidotris(diethylamido)Niobium(V), tert-butylimidotris(dimethylamido)Niobium(V), and tert-butylimidotris(ethylmethylamido)Niobium(V); and combinations of the foregoing.

43. The method of claim 19, wherein the Phosphorus source reactant is trimethyl phosphate (TMPO), diethyl phosphoramidate (DEPA), triethyl phosphate (TEPO), TMP, and combinations thereof.

44. The method of claim 16, wherein step a) and/or step b) is performed at a temperature above a melting point of the metal-containing chemical and at or below 200 degrees C.
